(19) United States
(12) Patent Application Publication
(10) Pub. No.: US 2022/0319635 A1
(43) Pub. Date: Oct. 6, 2022

(54) GENERATING MINORITY-CLASS EXAMPLES FOR TRAINING DATA

(71) Applicant: NEC Laboratories America, Inc., Princeton, NJ (US)

(72) Inventors: Renqiang Min, Princeton, NJ (US); Hans Peter Graf, South Amboy, NJ (US); Ligong Han, Edison, NJ (US)

(21) Appl. No.: 17/711,617

(22) Filed: Apr. 1, 2022

Related U.S. Application Data
(60) Provisional application No. 63/170,697, filed on Apr. 5, 2021.

(51) Int. Cl. G16B 20/30; G06N 3/08; G06N 3/04; G16B 40/20; G16B 30/00

(52) U.S. Cl. CPC G16B 20/30; G06N 3/08; G06N 3/0454; G16B 40/20; G16B 30/00

(57) ABSTRACT

Methods and systems for training a model include encoding training peptide sequences using an encoder model. A new peptide sequence is generated using a generator model. The encoder model, the generator model, and the discriminator model are trained to cause the generator model to generate new peptides that the discriminator mistakes for the training peptide sequences, including learning projection vectors with respective cross-entropy losses for binding sequences and non-binding sequences.

This application claims priority to U.S. Provisional Patent Application No. 63/170,697, filed on Apr. 5, 2021, incorporated herein by reference in its entirety.

### BACKGROUND

### Technical Field

The present invention relates to neural network training, and, more particularly, to generating minority-class examples for enhancing neural network training data.

### Description of the Related Art

Peptide-MHC (Major Histocompatibility Complex) protein interactions are involved in cell-mediated immunity, regulation of immune responses, and transplant rejection. While computational tools exist to predict a binding interaction score between an MHC protein and a given peptide, tools for generating new binding peptides with new specified properties from existing binding peptides are lacking.

### SUMMARY

A method for training a model includes encoding training peptide sequences using an encoder model. A new peptide sequence is generated using a generator model. The encoder model, the generator model, and the discriminator model are trained to cause the generator model to generate new peptides that the discriminator mistakes for the training peptide sequences, including learning projection vectors with respective cross-entropy losses for binding sequences and non-binding sequences.

A method for developing treatments includes training a generative adversarial network (GAN) model to generate binding peptide sequences relating to a major histocompatibility complex (MHC) protein associated with a virus pathogen or tumor. A new binding peptide sequence is generated using the trained GAN. A treatment for the virus pathogen or tumor is developed associated with the MHC protein using the new binding peptide sequence.

A system for training a model includes a hardware processor and a memory that stores a computer program. When executed by the hardware processor, the computer program causes the hardware processor to encode training peptide sequences using an encoder model, to generate a new peptide sequence using a generator model, and to train the encoder model, the generator model, and the discriminator model to cause the generator model to generate new peptides that the discriminator mistakes for the training peptide sequences, including learning projection vectors with respective cross-entropy losses for binding sequences and non-binding sequences.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram that illustrates binding between a peptide and a major histocompatibility complex (MHC), in accordance with an embodiment of the present principles;

FIG. 2 is a block diagram of a generative adversarial network (GAN) that can be trained to generate binding peptide sequences, in accordance with an embodiment of the present invention;

FIG. 3 is a block/flow diagram of a method for developing and administering a treatment for a given pathogen, in accordance with an embodiment of the present invention;

FIG. 4 is a block/flow diagram of a method for training a GAN to generate peptide sequences that can bind to a given MHC protein, in accordance with an embodiment of the present invention;

FIG. 5 is a block diagram of a neural network architecture of an exemplary peptide sequence discriminator, in accordance with an embodiment of the present invention;

FIG. 6 is a block diagram of a neural network architecture of an exemplary peptide sequence classifier, in accordance with an embodiment of the present invention;

FIG. 7 is a block diagram of a neural network architecture of an exemplary peptide sequence generator, in accordance with an embodiment of the present invention;

FIG. 8 is a diagram of a patient being treated using a treatment developed by generating a new binding peptide for a specific major histocompatibility complex, in accordance with an embodiment of the present invention;

FIG. 9 is a block diagram of a computing device that includes program code for training a model and generating new binding peptide sequences, in accordance with an embodiment of the present invention;

FIG. 10 is a diagram of an exemplary neural network architecture that may be used to implement one or more models, in accordance with an embodiment of the present invention; and

FIG. 11 is a diagram of an exemplary neural network architecture that may be used to implement one or more models, in accordance with an embodiment of the present invention.

### DETAILED DESCRIPTION OF PREFERRED EMBODIMENTS

Protein interactions between peptides and major histocompatibility complexes (MHCs) are involved in cell-mediated immunity, regulation of immune responses, and transplant rejection. Machine learning systems, including regression-based methods and neural network—based methos, may generate a prediction for a binding interaction score between an MHC protein and a given peptide. A machine learning system, as described herein, may generate new peptides with a strong binding interaction score with the MHC protein, based on one or more starting peptides.

Such generative systems may assume that the provided binding peptides are sufficient to train a generative model, such as a conditional generative adversarial network (GAN). However, new binding peptides may be generated, even when the provided training dataset is imbalanced, with a number of binding peptides being significantly smaller than the number of non-binding peptides.

The training dataset may be enhanced by introducing additional minority-class training examples. While the specific application to generating binding peptides is described in detail herein, it should be understood that the training dataset enhancement described herein may be applied to a variety of different applications where training data for a category to be identified may be scarce, such as in visual product defect classification and anomaly detection.

New binding peptides may be generated using a deep generative system that is trained using a dataset with both MHC-binding peptides and non-binding peptides. Instead of predicting binding scores of a predefined set of peptides, the conditional GAN is trained on MHC-binding peptides with dual class label projections and a generator with tempering softmax units.

A conditional Wasserstein GAN may be trained using a dataset that includes both binding and non-binding peptide sequences for an MHC. The conditional Wasserstein GAN may include a generator and a discriminator, with the generator being a deep neural network that transforms a sampled latent code vector z and a sampled label y to a generated peptide sequence.

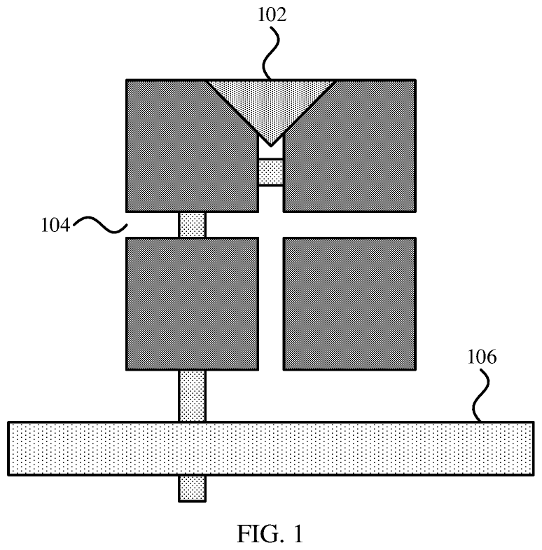

Referring now to FIG. 1, a diagram of a peptide-MHC protein bond is shown. A peptide 102 is shown as bonding with an MHC protein 104, with complementary two-dimensional interfaces of the figure suggesting complementary shapes of these three-dimensional structures. The MHC protein 104 may be attached to a cell surface 106.

An MHC is an area on a DNA strand that codes for cell surface proteins that are used by the immune system. MHC molecules are used by the immune system and contribute to the interactions of white blood cells with other cells. For example, MHC proteins impact organ compatibility when performing transplants and are also important to vaccine creation.

A peptide, meanwhile, may be a portion of a protein. When a pathogen presents peptides that are recognized by a MHC protein, the immune system triggers a response to destroy the pathogen. Thus, by finding peptide structures that bind with MHC proteins, an immune response may be intentionally triggered, without introducing the pathogen itself to a body. In particular, given an existing peptide that binds well with the MHC protein 104, a new peptide 102 may be automatically identified according to desired properties and attributes.

Although the present principles are described with specific focus on the generation of binding peptides, they may be readily extended to include continuous binding affinity predictions of peptide sequences, naturally processed peptide predictions of peptide sequences, T-cell epitope predictions of peptide sequences, etc. Varying the application involves providing different supervision signals for optimizing the cross-entropy loss terms, described in greater detail below.

Furthermore, the present principles are not limited to binding peptide generation, but may be extended to generate other minority-class examples with other applications. For example, minority-class product images may be generated for product inspection and anomaly detection. For such tasks, the input training data may include images, and the generator architecture may be altered to accommodate that input format.

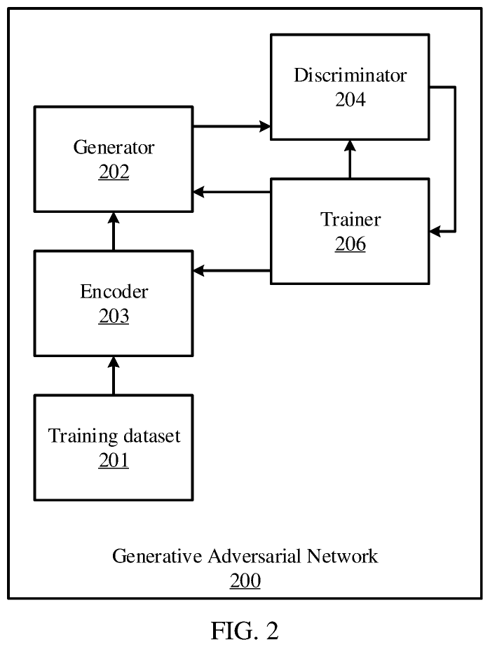

Referring now to FIG. 2, an exemplary GAN 200 is shown. The GAN 200 includes a generator 202 and a discriminator 204. The generator 202 generates training dataset candidates, while the discriminator 204 attempts to distinguish between the generated candidates and true samples from a provided training dataset 201. An encoder 203 converts the sequences of the training dataset into vectors in an embedded space. The encoder may use block substitution or a pre-trained amino acid embedding scheme to convert the amino acid sequence into, e.g., a feature representation matrix, with each column of the matrix corresponding to an amino acid. The encoder 203 and the generator 202 may be trained together to fool the discriminator.

The generator 202 is trained to increase the error rate of the discriminator 204, while the discriminator 204 is trained to decrease its error rate in identifying the generated candidates. A trainer 206 uses a loss function to perform training for the generator 202 and the discriminator 204. In a Wasserstein GAN, the loss function may be based on the Wasserstein metric.

In the context of peptide generation, the training dataset 201 may include both binding and nonbinding peptide sequences that interact with an MHC. The generator 202 may be a deep neural network, which transforms a sampled latent code vector z from a multivariate unit-variance Gaussian distribution and a sampled binding class label (e.g., 1 for “binding” and 0 for “non-binding”) to a peptide feature representation matrix, with each column corresponding to an amino acid.

The discriminator 204 may be a deep neural network with convolutional layers and fully connected layers between an input representation layer and an output layer that outputs a scalar value. The parameters of the discriminator 204 may be updated to distinguish generated peptide sequences from sampled peptide sequences in the training dataset 201. The parameters of the generator 202 are updated to fool the discriminator 204.

A dual-projection GAN can be used to simultaneously learn two projection vectors, with two cross-entropy losses for each class (e.g., “binding” and “non-binding”). This is equivalent to maximizing the mutual information between generated data examples and their associated labels, with one loss discriminating between real binding/non-binding peptides in the training data and real non-binding/binding peptides in the training data, and the other loss discriminating between generating binding/non-binding peptides and generated non-binding/binding peptides. The generator 202 may be updated to minimize these two cross-entropy losses for each class.

A non-negative scalar weight λ(x) may be learned for each data point x associated with the two cross-entropy losses, balancing the discriminator loss. A penalty term of −0.5 log(λ(x)) may be added to penalize large values of λ(x). Data-label pairs may be denoted as {xᵢ, yᵢ}ᵢ₌₁ⁿ⊆x×y, drawn from a joint distribution P_{xy}, where x is a peptide sequence and y is a label. The generator 202 is trained to transform samples z˜P_{z} from a canonical distribution conditioned on labels to match the real data distributions, with real distributions being denoted as P and with generated distributions being denoted as Q. The discriminator 204 learns to distinguish samples drawn from the joint distribution P_{xy} and Q_{xy}.

Discriminator and generator loss terms may be written as the following objectives:

L_{D}= [symbol] _{x,y˜P}_{_{XY}} [symbol] (−\tilde{D}(x,y))+ [symbol] _{z˜P}_{_{z,y}}_{˜Q}_{_{y}} [symbol] (\tilde{D}(G(z,y),y))

LG= [symbol] z˜_{P}_{_{z,y}}_{˜Q}_{_{y}}A(−\tilde{D}(G(z,y),y))

where [symbol] (·) is an activation and \tilde{D} is the discriminator's output before activation. The activation function may be [symbol] (t)=softplus(t)=log(1+e^{t}). With this activation function, the logit of an optimal discriminator can be decomposed in two ways:

$${\tilde{D}}^{*} ( x , y ) = log ( \frac{P ( x )}{Q ( x )} ) + log ( \frac{P ( y \vert x )}{Q ( y \vert x )} )$$

$${\tilde{D}}^{*} ( x , y ) = log ( \frac{P ( x \vert y )}{Q ( x \vert y )} ) + log ( \frac{P ( y )}{Q ( y )} )$$

The logic of a projection discriminator can be derived as:

\tilde{D}(x,y)=v_{y}^{T}ϕ(x)+ψ(ϕ(x))

where ϕ(·) is the image embedding function, v_{y}, is an embedding of class y, and ψ collects residual terms. The term v_{y} can be expressed as a difference of real and generated class embeddings, v_{y}=V_{y}^{P}−v_{y}^{q}.

Thus, a projection discriminator can tie the parameters V_{y}^{P} and v_{y}^{q} to a single v_{y}. Tying embeddings can turn the problem of learning categorical decision boundaries into learning a relative translation vector for each class, which is a simpler process. Without loss of generality, the term ψ(·) may be assumed to be a linear function v_{ψ}. The softplus function may be approximated by ReLU=max(0,.), which produces a large loss when x⁺ and x⁻ are misclassified. Thus, learning can be performed by alternating the steps:

Discriminator: Align (v_{y}+v_{ψ}) with (ϕ(x⁺)−ϕ(x⁻))
Generator: Move ϕ(x⁻) along (v_{y}+V_{ψ})

By tying the parameters, the GAN can directly perform data matching without explicitly enforcing label matching, aligning Q(x|y) with P (x|y).

The term v_{y} should recover the difference between the underlying v_{y}^{p} and v_{y}^{q}, but to explicitly enforce that property, the class embeddings may be separated out, and V^{P} and v^{q} may be used to learn conditional distributions p(y|x) and q(y|x), respectively. This may be done with the softmax function, and cross-entropy losses may be expressed as:

$${L}_{m i}^{p} = - {v}_{y}^{p T} ϕ ( {x}^{+} ) + log {\sum }_{{y}^{′}} {e}^{{v}_{{y}^{′}}^{p T} ϕ ( {x}^{+} )}$$

$${L}_{m i}^{q} = - {v}_{y}^{q T} ϕ ( {x}^{-} ) + log {\sum }_{{y}^{′}} {e}^{{v}_{{y}^{′}}^{p T} ϕ ( {x}^{-} )}$$

$${L}_{D}^{P 2} = {L}_{D} ( \tilde{D} ) + {L}_{m i}^{p} + {L}_{m i}^{q}$$

$${L}_{G}^{P 2} = {L}_{G} ( \tilde{D} )$$

$$\tilde{D} = {( {v}_{u}^{p} - {v}_{u}^{q} )}^{T} ϕ ( x ) + ψ ( ϕ ( x ) )$$

where p and q correspond to conditional distribution or loss function using real/generated binding peptides, the terms V_{y}^{P} and v_{y}^{q} represent embeddings of the real and generated samples, respectively, ϕ(·) is an embedding function, ψ(·)collects residual terms, and x⁺˜P_{X} and x⁻˜Qₓ are real and generated sequences (with P and Q being the respective real and generated distributions), and y is a data label. The classifiers V^{P} and v^{q} are trained on real data and generated data, respectively. The discriminator loss L_{D}^{P2} and generator loss L_{G}^{P2} trained as above. Both L_{D} (\tilde{D}) and Lₘᵢ^{P} include the parameter V^{P}, while L_{D} (\tilde{D}) and Lₘᵢ^{q} both include V^{q}.

Data matching and label matching may be weighted by the model. A gate may be added between the two losses:

L_{D}^{P2w}=L_{D}λ(Lₘᵢ^{P}+Lₘᵢ^{q})

The definition of A changes the behavior of the system. Variants may include exponential decay, scalar valued, and amortized models. For example, A may be defined as a decaying factor,

$$λ = {e}^{- \frac{t}{T}} ,$$

where t is a training iteration and T is a maximum number of training iterations.

In a scalar valued embodiment, if λ≥0 is a learnable parameter, initialized as 1, class separation may be enforced as long as λ≥0. A penalty term may be used:

$${L}_{D}^{P 2 sp} = {L}_{D} + λ ( {L}_{m i}^{p} + {L}_{m i}^{q} ) - \frac{1}{2} log λ$$

In an amortized embodiment, amortized homoscedastic weights may be learned for each data point. The term λ(x)≥0 would then be a function of x producing per-sample weights. A penalty can be added. When loss terms involve non-linearity in the mini-batch expectation, any type of linearization may be applied.

Softmax may be used in the last output layer of the generator 202, with entropy regularization being used to implicitly control the temperature in the tempering softmax units. In a forward pass, a straight-through estimator may be used to output discrete amino acid sequences (e.g., peptides) with “binding” or “non-binding” labels. In the backward pass, the temperatures may be used to facilitate continuation gradient calculations. At the beginning of training, a smaller penalty coefficient may be set for entropy regularization to encourage more uniform amino acid emission probability distributions. Later in training, a larger penalty coefficient may be used for entropy regularization to encourage amino acid emission probability distributions with more peaks.

Besides updating the discriminator 204 and generator 202 in a weighted framework, an encoder may be trained to map an input peptide sequence x to a latent embedding code space z. The aggregated latent codes of the input peptide sequences may be enforced to follow a multivariate unit-variance Gaussian distribution, by minimizing a kernel maximum mean discrepancy regularization term. Each embedding code z is fed into the generator 202 to reconstruct the original peptide sequence x, and the encoder and the generator 202 may be updated by minimizing a cross-entropy loss as the reconstruction error.

During the training, m binding peptide sequences may be randomly sampled from the training set 201. A convex combination of the latent codes of the m peptides may be calculated with randomly sampled coefficients, where 2≤m≤K and K is a user-specified hyperparameter. A convex combination may be a positive-weighted linear combination with the sum of the weights equal to 1. The generator 202 generates a binding peptide, and the encoder and generator 202 are updated so that the classifier q (y|x) for the binding class will correctly classify the generated peptide and so the discriminator 204 will classify it as real data.

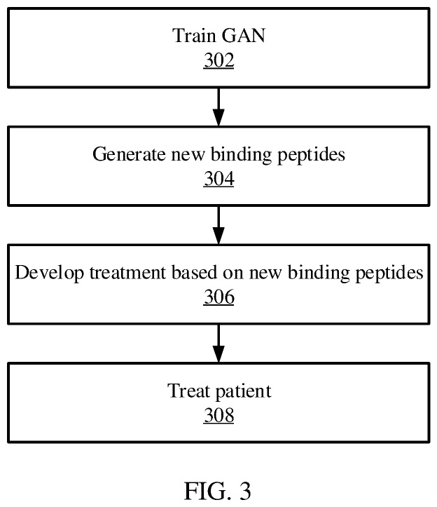

Referring now to FIG. 3, a method for developing treatments is shown. Block 302 trains the GAN 200 to generate new binding peptide sequences, using a training dataset that includes both binding and non-binding peptides. From the trained GAN 200, the generator 202 can then generate new binding peptides for a given MHC protein of a pathogen in block 304. Having identified peptides that bind well to the MHC protein of the pathogen, block 306 generates a treatment based on the peptides. Block 308 then treats a patient using the developed treatment, for example by administering a drug that includes the identified peptides, which bind to the MHC protein of the pathogen and encourage the patient's immune system to target the pathogen.

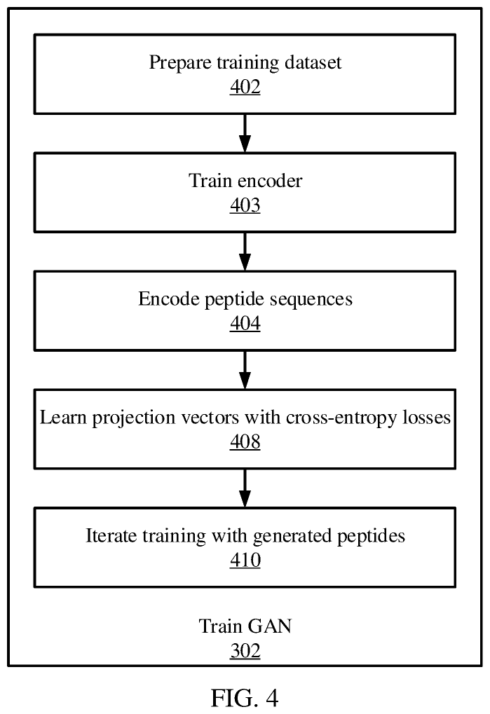

Referring now to FIG. 4, additional detail on the training of block 302 is shown. Block 402 generates a training dataset. The training dataset may include a set of peptide sequences, each of which may be labeled as binding or non-binding with respect to an MHC protein. Block 403 trains an encoder to convert peptide sequences into a vector embedded in a latent space. As noted above, the encoder maps input peptide sequence x to the space z, including minimizing a kernel maximum mean discrepancy regularization term to enforce a multivariate unit-variance Gaussian distribution. The training of the encoder is performed alongside training the generator 202, as the reconstruction error is used to help minimize a cross-entropy loss.

Block 404 uses the trained encoder to encode the peptide sequences of the training dataset as vectors. These vectors, in turn, as used as inputs to the generator 202. Block 408 learns dual projection vectors of the GAN 200. The GAN objective function is optimized with two cross-entropy losses for each classes and with data-specific adaptive weights balancing the discriminator loss and the cross-entropy losses. The generator 202 is updated with tempering softmax outputs to minimize the cross-entropy losses. This training across blocks 403, 404, and 408 is iterated in block 410, with convex combinations of binding sequence embeddings being used to generate binding peptides. The encoder and the generator 202 are updated to fool the discriminator 204 and the classifier. Iteration stops when a maximum number of iterations has been reached.

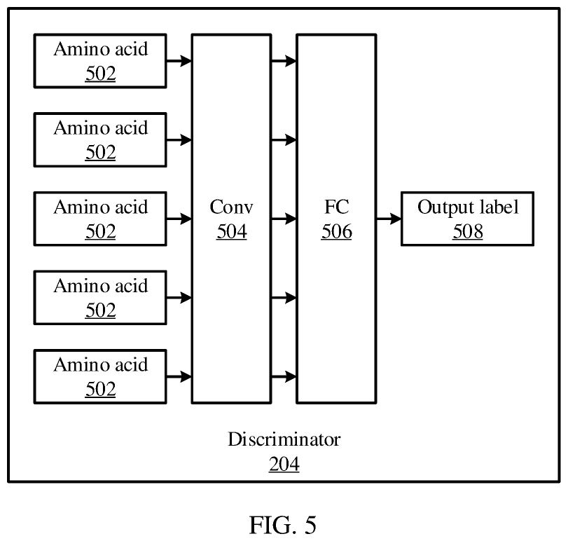

Referring now to FIG. 5, an exemplary architecture for the discriminator 204 is shown. A peptide sequence is input as a series of embedded amino acids 502, which are processed by a convolutional layer 504 and one or more fully connected layers 506. The output of the final fully connected layer is a label, indicating whether the input amino acids 502 represent a “real” sequence, present within the training dataset 201, or a sequence that was generated by the generator 202.

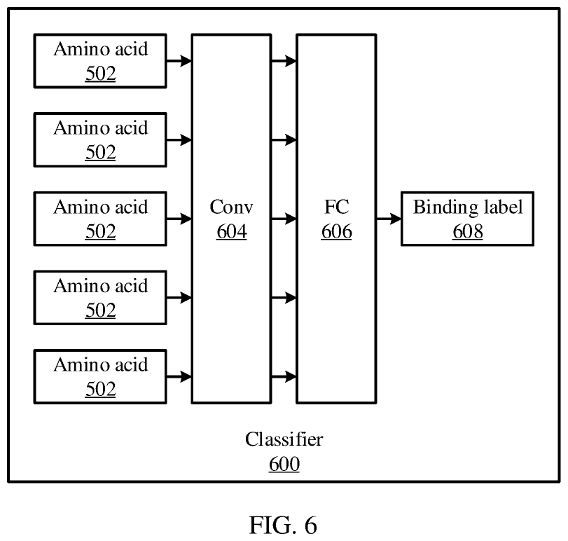

Referring now to FIG. 6, an exemplary architecture for the classifier is shown. As with the discriminator 204, a peptide sequence is input as a series of embedded amino acids 502. The input amino acids 502 are processed by a convolutional layer 604 and one or more fully connected layers 606, trained to identify whether a given peptide sequence binds with an MHC protein. The output of the final fully connected layer is a label, indicating whether the input amino acids 502 represent a binding sequence or a non-binding sequence.

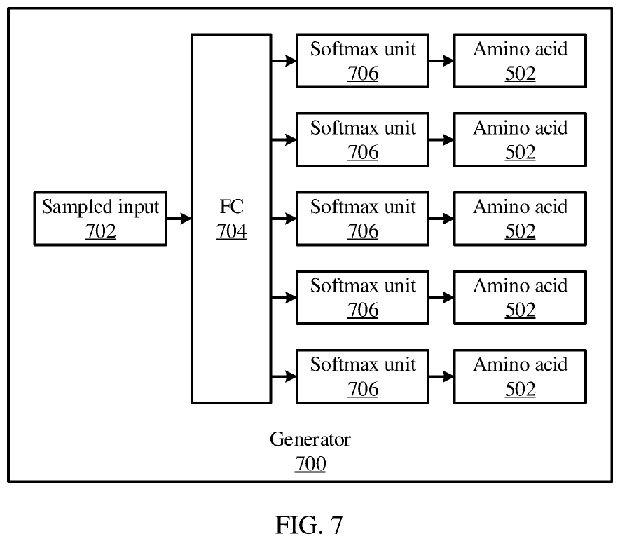

Referring now to FIG. 7, an exemplary architecture for the generator 700 is shown. Block 702 samples a random noise vector z and a class y as an input to the generator. This vector may be sampled from a multivariate Gaussian distribution with zero mean and unit diagonal variance, and the binding class label may be fixed.

The sampled vector and class are processed by one or more fully connected layers 704, which are trained to convert the input into a representation of a peptide sequence. A series of output tempering softmax units 706 processes the output of the fully connected layer(s) 704, generating respective amino acids 502 that, together, form a peptide sequence.

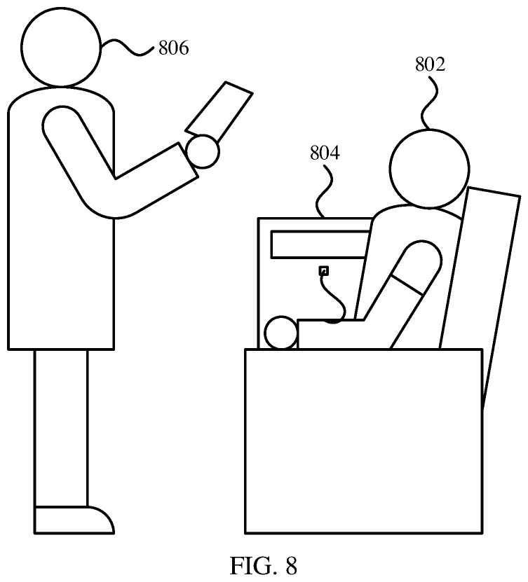

Referring now to FIG. 8, treatment of a patient 802 is illustrated. A treatment system 804 administers a treatment that is based on a peptide sequence generated by the GAN 200. In particular, a binding peptide may be generated that corresponds to a pathogen or tumor of the patient 102. This binding peptide may be used as part of a treatment that is provided to the patient 102, where the peptide binds to an MHC protein on the pathogen or the tumor cells, helping the patient's autoimmune system identify and remove the pathogen or tumor.

The administration of the treatment may be overseen by a medical professional 806, who can help connect the treatment system 804. The medical professional 806 may also be involved in the identification of the pathogen or tumor, using diagnostic tools to isolate MHC proteins to be used in identifying binding peptides.

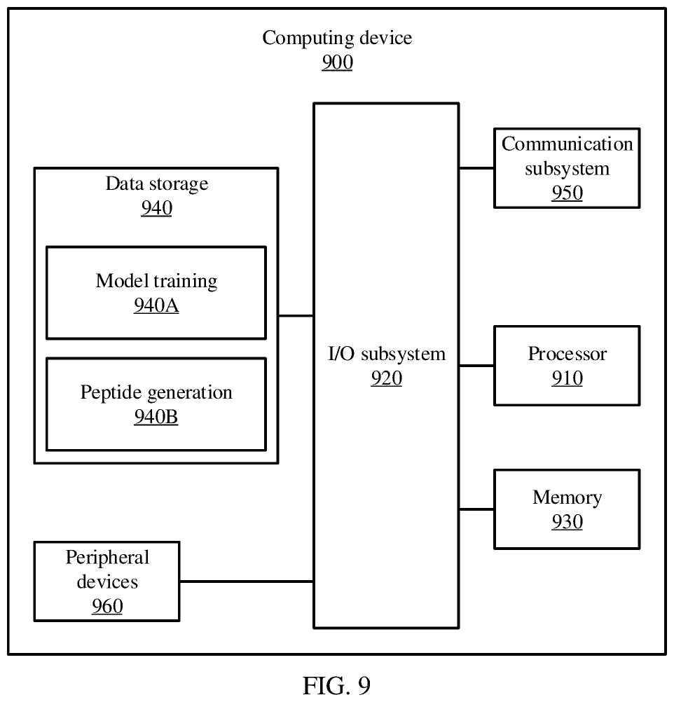

Referring now to FIG. 9, an exemplary computing device 900 is shown, in accordance with an embodiment of the present invention. The computing device 900 is configured to perform classifier enhancement.

The computing device 900 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor- based system, and/or a consumer electronic device. Additionally or alternatively, the computing device 900 may be embodied as a one or more compute sleds, memory sleds, or other racks, sleds, computing chassis, or other components of a physically disaggregated computing device.

As shown in FIG. 9, the computing device 900 illustratively includes the processor 910, an input/output subsystem 920, a memory 930, a data storage device 940, and a communication subsystem 950, and/or other components and devices commonly found in a server or similar computing device. The computing device 900 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 930, or portions thereof, may be incorporated in the processor 910 in some embodiments.

The processor 910 may be embodied as any type of processor capable of performing the functions described herein. The processor 910 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 930 may be embodied as any type of volatile or non-volatile memory or data storage capable of performing the functions described herein. In operation, the memory 930 may store various data and software used during operation of the computing device 900, such as operating systems, applications, programs, libraries, and drivers. The memory 930 is communicatively coupled to the processor 910 via the I/O subsystem 920, which may be embodied as circuitry and/or components to facilitate input/output operations with the processor 910, the memory 930, and other components of the computing device 900. For example, the I/O subsystem 920 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 920 may form a portion of a system-on-a-chip (SOC) and be incorporated, along with the processor 910, the memory 930, and other components of the computing device 900, on a single integrated circuit chip.

The data storage device 940 may be embodied as any type of device or devices configured for short-term or long-term storage of data such as, for example, memory devices and circuits, memory cards, hard disk drives, solid state drives, or other data storage devices. The data storage device 940 can store program code 940A for model training and program code 940B for generating binding peptides. The communication subsystem 950 of the computing device 900 may be embodied as any network interface controller or other communication circuit, device, or collection thereof, capable of enabling communications between the computing device 900 and other remote devices over a network. The communication subsystem 950 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 900 may also include one or more peripheral devices 960. The peripheral devices 960 may include any number of additional input/output devices, interface devices, and/or other peripheral devices. For example, in some embodiments, the peripheral devices 960 may include a display, touch screen, graphics circuitry, keyboard, mouse, speaker system, microphone, network interface, and/or other input/output devices, interface devices, and/or peripheral devices.

Of course, the computing device 900 may also include other elements (not shown), as readily contemplated by one of skill in the art, as well as omit certain elements. For example, various other sensors, input devices, and/or output devices can be included in computing device 900, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 900 are readily contemplated by one of ordinary skill in the art given the teachings of the present invention provided herein.

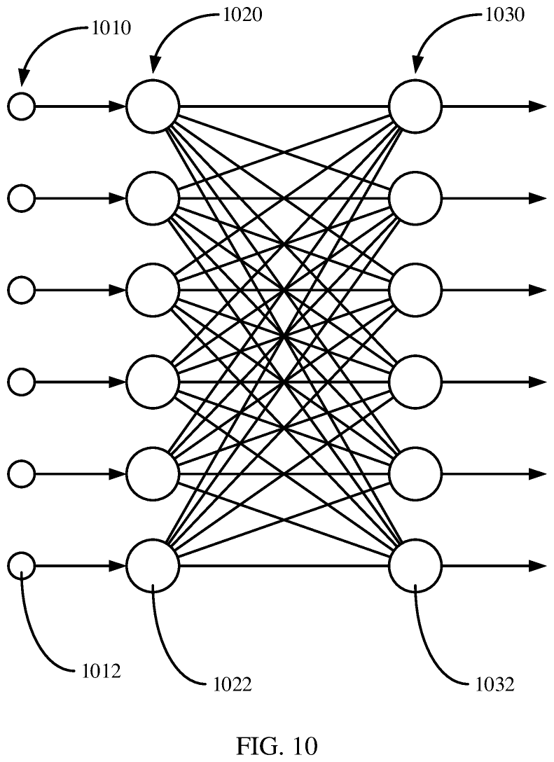

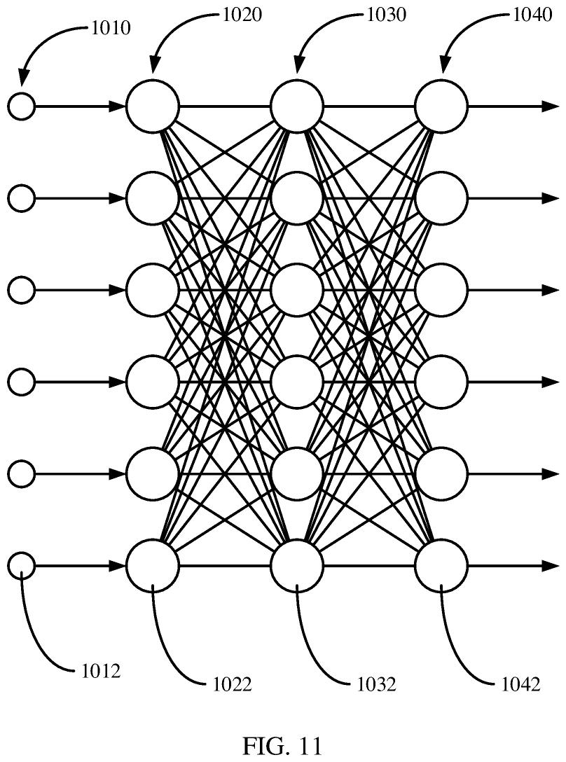

Referring now to FIGS. 10 and 11, exemplary neural network architectures are shown, which may be used to implement parts of the present models. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be outputted.

The empirical data, also known as training data, from a set of examples can be formatted as a string of values and fed into the input of the neural network. Each example may be associated with a known result or output. Each example can be represented as a pair, (x,y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 1020 of source nodes 1022, and a single computation layer 1030 having one or more computation nodes 1032 that also act as output nodes, where there is a single computation node 1032 for each possible category into which the input example could be classified. An input layer 1020 can have a number of source nodes 1022 equal to the number of data values 1012 in the input data 1010. The data values 1012 in the input data 1010 can be represented as a column vector. Each computation node 1032 in the computation layer 1030 generates a linear combination of weighted values from the input data 1010 fed into input nodes 1020, and applies a non-linear activation function that is differentiable to the sum. The exemplary simple neural network can perform classification on linearly separable examples (e.g., patterns).

A deep neural network, such as a multilayer perceptron, can have an input layer 1020 of source nodes 1022, one or more computation layer(s) 1030 having one or more computation nodes 1032, and an output layer 1040, where there is a single output node 1042 for each possible category into which the input example could be classified. An input layer 1020 can have a number of source nodes 1022 equal to the number of data values 1012 in the input data 1010. The computation nodes 1032 in the computation layer(s) 1030 can also be referred to as hidden layers, because they are between the source nodes 1022 and output node(s) 1042 and are not directly observed. Each node 1032, 1042 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the inputted data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 1032 in the one or more computation (hidden) layer(s) 1030 perform a nonlinear transformation on the input data 1012 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method of training a model, comprising:
encoding training peptide sequences using an encoder model;
generating a new peptide sequence using a generator model; and
training the encoder model, the generator model, and the discriminator model to cause the generator model to generate new peptides that the discriminator mistakes for the training peptide sequences, including learning projection vectors with respective cross-entropy losses for binding sequences and non-binding sequences.

2. The computer-implemented method of claim 1, wherein the generator model outputs amino acid representations using a plurality of tempering softmax output units.

3. The computer-implemented method of claim 1, wherein generating the new peptide sequence includes sampling a multivariate unit-variate Gaussian distribution as input to the generator.

4. The computer-implemented method of claim 1, wherein the cross-entropy losses include:
$${L}_{m i}^{p} = - {v}_{y}^{p T} ϕ ( {x}^{+} ) + log {\sum }_{{y}^{′}} {e}^{{v}_{{y}^{′}}^{p T} ϕ ( {x}^{+} )}$$
$${L}_{m i}^{q} = - {v}_{y}^{q T} ϕ ( {x}^{-} ) + log {\sum }_{{y}^{′}} {e}^{{v}_{{y}^{′}}^{p T} ϕ ( {x}^{-} )}$$
where p corresponds to training peptide sequences, q corresponds to peptide sequences generated by the generator model, v_{y}^{p} represents embeddings of the training peptide sequences and generated peptide sequences, v_{y}^{p} represents embeddings of the peptide sequences generated by the generator model, ϕ(·) is an embedding function, and x⁺˜P_{X} and x⁻˜Qₓ are respective training and generated sequences, with P and Q being respective training and generated distributions.

5. The method of claim 1, wherein the encoder model embeds peptide sequences from a training dataset into vectors during training.

6. The method of claim 5, wherein training the encoder model includes minimizing a kernel maximum mean discrepancy regularization term.

7. The method of claim 5, wherein the training dataset includes binding peptide sequences and nonbinding peptide sequences relative to a major histocompatibility complex.

8. The method of claim 5, wherein the generator transforms a binding class label from the encoder and a sampled latent code vector into a peptide feature representation matrix, with each column of the matrix corresponding to an amino acid.

9. The method of claim 1, wherein training the encoder model, the generator model, and the discriminator model uses a loss function that is based on a Wasserstein metric.

10. A computer-implemented method for developing treatments, comprising:
training a generative adversarial network (GAN) model to generate binding peptide sequences relating to a major histocompatibility complex (MHC) protein associated with a virus pathogen or tumor;
generating a new binding peptide sequence using the trained GAN;
developing a treatment for the virus pathogen or tumor associated with the MHC protein using the new binding peptide sequence.

11. The method of claim 10, further comprising treating a person for the virus pathogen or tumor using the developed treatment.

12. A system for training a model, comprising:
a hardware processor; and
a memory that stores a computer program, which, when executed by the hardware processor, causes the hardware processor to:
encode training peptide sequences using an encoder model;
generate a new peptide sequence using a generator model; and
train the encoder model, the generator model, and the discriminator model to cause the generator model to generate new peptides that the discriminator mistakes for the training peptide sequences, including learning projection vectors with respective cross-entropy losses for binding sequences and non-binding sequences.

13. The system of claim 12, wherein the generator model outputs amino acid representations using a plurality of tempering softmax output units.

14. The system of claim 12, wherein the computer program further causes the hardware processor to sample a multivariate unit-variate Gaussian distribution as input to the generator.

15. The system of claim 12, wherein the cross-entropy losses include:
$${L}_{m i}^{p} = - {v}_{y}^{p T} ϕ ( {x}^{+} ) + log {\sum }_{{y}^{′}} {e}^{{v}_{{y}^{′}}^{p T} ϕ ( {x}^{+} )}$$
$${L}_{m i}^{q} = - {v}_{y}^{q T} ϕ ( {x}^{-} ) + log {\sum }_{{y}^{′}} {e}^{{v}_{{y}^{′}}^{p T} ϕ ( {x}^{-} )}$$
where p corresponds to training peptide sequences, q corresponds to peptide sequences generated by the generator model, v_{y}^{p} represents embeddings of the training peptide sequences and generated peptide sequences, v_{y}^{p} represents embeddings of the peptide sequences generated by the generator model, ϕ(·) is an embedding function, and x⁺˜P_{X} and x⁻˜Qₓ are respective training and generated sequences, with P and Q being respective training and generated distributions.

16. The system of claim 12, wherein the encoder model embeds peptide sequences from a training dataset into vectors during training.

17. The system of claim 16, wherein the computer program further causes the hardware processor to minimize a kernel maximum mean discrepancy regularization term to train the encoder model.

18. The system of claim 17, wherein the training dataset includes binding peptide sequences and nonbinding peptide sequences relative to a major histocompatibility complex.

19. The system of claim 12, wherein the generator transforms a binding class label from the encoder and a sampled latent code vector into a peptide feature representation matrix, with each column of the matrix corresponding to an amino acid.

20. The system of claim 12, wherein the computer program further causes the hardware processor to use a loss function that is based on a Wasserstein metric to train the encoder model, the generator model, and the discriminator model.
